(19) United States
(12) Patent Application Publication
(10) Pub. No.: US 2026/0285793 A1
(43) Pub. Date: Sep. 24, 2026

(54) METHOD FOR SYNTHESIZING CYCLIC KETONE COMPOUND FROM DIOL AND KETONE

(71) Applicant: Zhejiang Sainon Chemical Co., Ltd., Quzhou (CN)

(72) Inventors: Ji'an FAN, Quzhou (CN); Manna HU, Quzhou (CN); Shuqun XU, Quzhou (CN); Jianhui LI, Quzhou (CN)

(73) Assignee: Zhejiang Sainon Chemical Co., Ltd., Quzhou (CN)

(21) Appl. No.: 19/312,352

(22) Filed: Aug. 28, 2025

(30) Foreign Application Priority Data
Mar. 18, 2025 (CN) 202510314953.3

(51) Int. Cl. C07C 45/66; B01J 21/18; B01J 23/745; B01J 23/75; B01J 23/755; C07C 45/79

(52) U.S. Cl. CPC C07C 45/66; B01J 21/18; B01J 23/745; B01J 23/75; B01J 23/755; C07C 45/79; C07C 2601/08; C07C 2601/14; C07C 2601/18

(57) ABSTRACT

A method for synthesizing a cyclic ketone compound from a diol and a ketone is provided. The method includes: employing a ketone and a diol as reaction substrates, and cyclizing by coupling the α-carbon of the carbonyl group in the ketone with the α-carbon of the hydroxyl group in the diol under catalysis of a transition metal catalyst, to form a novel cyclic ketone compound. In the method for synthesizing the cyclic ketone compound from the diol and the ketone, after adding the reactants, the reaction proceeds without requiring an external solvent or high-pressure hydrogen, making the process easy to implement with a straightforward preparation method. The method exhibits broad applicability, with both high conversion rates of the diol and the ketone, and excellent selectivity toward the target cyclic ketone compound.

### CROSS REFERENCE TO THE RELATED APPLICATIONS

This application is based upon and claims priority to Chinese Patent Application No. 202510314953.3, filed on Mar. 18, 2025, the entire contents of which are incorporated herein by reference.

### TECHNICAL FIELD

The present invention belongs to the technical field of organic synthesis, and particularly relates to a method for synthesizing a cyclic ketone compound from a diol and a ketone.

### BACKGROUND

Cyclic ketone compounds refer to those compounds containing a carbocyclic ring and a carbonyl (C═O) functional group. Depending on the nature of the hydrocarbon group in the molecule, cyclic ketone compounds may be further classified into, including but not limited to alicyclic ketones and aromatic ketones. In the field of organic chemistry, cyclic ketone compounds serve as important organic intermediates and are widely used in pharmaceuticals, agrochemicals, dyes, fragrances, and related technical fields. Cyclopentanone can be used in the flavor and fragrance industry for producing methyl dihydrojasmonate, magnone and other products. It is also applicable in the agrochemical field for manufacturing insecticides, herbicides and similar products. Further applications include the electronics industry (e.g., developing solutions) and the pharmaceutical sector for producing anti-anxiety medications and other therapeutic agents. Cyclohexanone, as an important chemical product, serves as a key raw material for producing adipic acid and caprolactam, while also functioning as a significant industrial solvent. Currently, various synthetic methods exist for cyclic ketone compounds, but the methods generally suffer from harsh reaction conditions, low yields, and poor purity.

As disclosed in Chinese Patent Application Publication No. CN118666655A, a one-step method for preparing cyclopentanone from cyclopentene has been reported. However, this method requires the introduction of an oxidizing agent (H₂O₂ aqueous solution), which tends to generate hazardous byproducts during the process. Additionally, the subsequent treatment of waste liquid is challenging, and the reaction imposes stringent operational requirements. Furthermore, the process suffers from low conversion rates of cyclopentene.

Additionally, Chinese Patent Application Publication No. CN116262689A discloses a method for preparing cyclohexanone. The method employs cyclohexyl carboxylate as feedstock, which undergoes direct dehydrogenation to form cyclohexanone in the presence of a supported noble metal catalyst and water. However, this reaction requires nitrogen gas introduction and demands stringent reaction conditions.

Therefore, developing an efficient, environmentally benign, and cost-effective method for preparing cyclic ketone compounds is of significant importance.

### SUMMARY

An objective of the present invention is to provide a method for synthesizing a cyclic ketone compound from a diol and a ketone, aiming to solve the aforementioned technical problems.

In view of the foregoing, the present invention provides a method for synthesizing a cyclic ketone compound from a diol and a ketone, the method includes: employing a ketone and a diol as reaction substrates, and cyclizing by coupling the α-carbon of the carbonyl group in the ketone with the α-carbon of the hydroxyl group in the diol under catalysis of a transition metal catalyst, to form a novel cyclic ketone compound.

Further, the method includes the steps of:
S1. adding at least one diol into a cleaned reactor;
S2. adding at least one ketone into the reactor in S1;
S3. adding the transition metal catalyst into the reactor in S2; and
S4. sealing the reactor, raising a temperature of the reactor to 80-400° C., reacting under stirring for 0.5-8 h, and then filtering a reaction product to obtain the cyclic ketone compound.

Further, the diol is selected from at least one of ethylene glycol, 1,2-propanediol, 1,3-propanediol, 1,2-butanediol, 1,4-butanediol, 1,2-pentanediol, 1,5-pentanediol, 1,2-hexanediol, 1,6-hexanediol, 1,2-heptanediol, 1,7-heptanediol, 1,2-octanediol, 1,8-octanediol, 1,2-nonanediol, 1,9-nonanediol, 1,2-decanediol, and 1,10-decanediol.

Further, the ketone in the reaction substrates is selected from at least one of acetone, butanone, 2-pentanone, 2-hexanone, 2-heptanone, 2-octanone, 2-nonanone, 2-decanone, and 2-undecanone.

Further, the transition metal catalyst is a supported transition metal catalyst.

Further, the supported transition metal catalyst includes at least one transition metal element selected from Group VIIB, Group VIII, Group IB, and Group IIB of the periodic table, wherein the transition metal is a non-precious metal element.

Further, in S4, a temperature of the reactor is 150-260° C.

Further, in the added reaction substrates, a molar ratio of the diol to the ketone is in a range of (1:50)≤(n1:n2)≤(20:1), wherein the number of moles of the diol is n1, and the number of moles of the ketone is n2.

Further, the reactor for synthesis is a high-pressure reactor.

Further, the prepared cyclic ketone compound includes cyclopentanone and cyclopentanone with R-group substituents on the side chain, cyclohexanone and cyclohexanone with R-group substituents on the side chain, cycloheptanone and cycloheptanone with R-group substituents on the side chain, cyclononanone and cyclononanone with R-group substituents on the side chain, as well as higher-membered cyclic ketones and derivatives with R-group substituents on the side chain, wherein R is a C1-C9 alkyl group.

The advantageous effects of the present invention include: the present invention provides a method for synthesizing a cyclic ketone compound from a diol and a ketone. The method includes adding a supported transition metal catalyst, diol, and ketone into a high-pressure reactor, controlling reaction temperature and agitation speed, via a highly efficient catalytic condensation-coupling reaction between the ketone and a small-molecule alcohol, to obtain a target cyclic ketone with high selectivity. In the method for synthesizing the cyclic ketone compound from the diol and the ketone according to the present invention, after adding the reactants, the reaction proceeds without requiring an external solvent or high-pressure hydrogen, making the process easy to implement with a straightforward preparation method. Furthermore, the method for synthesizing the cyclic ketone compound from the diol and the ketone according to the present invention is applicable to a broad range of diol and ketone substrates, with conversion rates exceeding 90% for both the diol and the ketone. The target cyclic ketone compound is obtained with a selectivity of at least 90%, and the process demonstrates long-term operational stability, exhibiting excellent potential for industrial application.

### BRIEF DESCRIPTION OF THE DRAWINGS

FIG. 1 is a schematic diagram illustrating a process for synthesizing 3-methylcyclopentanone from acetone and 1,2-propanediol;

FIG. 2 is a schematic diagram illustrating a process for synthesizing cycloheptanone from acetone and 1,4-butanediol;

FIG. 3 is a schematic diagram illustrating a process for synthesizing cyclononanone from acetone and 1,6-hexanediol;

FIG. 4 is a schematic diagram illustrating a process for synthesizing cyclohexanone from acetone and 1,3-propanediol;

FIG. 5 is a schematic diagram illustrating a process for synthesizing 2-methylcycloheptanone from butanone and 1,4-butanediol; and

FIG. 6 is a schematic diagram illustrating a process for synthesizing 2-ethylcycloheptanone from 2-pentanone and 1,4-butanediol.

### DETAILED DESCRIPTION OF THE EMBODIMENTS

The technical solutions of the present application will be described clearly below with reference to examples and accompanying drawings. It should be understood that the described examples represent only part of, rather than all, the examples of the present application. All other examples obtained by those of ordinary skill in the art based on the examples of the present application shall fall within the protection scope of the present application.

It shall be understood that the term “and/or” as used herein merely describes an associative relationship between related objects, indicating that three types of relationships may exist. For example, “A and/or B” may represent: (i) A alone, (ii) both A and B together; or (iii) B alone. Additionally, the character “/” as used herein generally indicates an ‘or’ relationship between the preceding and succeeding associated elements.

In the description of the specification, it should be understood that terms such as “substantially”, “approximately”, “about”, “nearly”, “generally”, and “essentially” as used in the claims and examples of the present invention refer to reasonable ranges within normal operating or tolerance limits, rather than denoting precise values.

It should be noted that the terminology used in the examples of the present invention is for the purpose of describing particular examples only and is not intended to limit the scope of the present invention. In the present application, the terms “comprise”, “include”, or any variations thereof are intended to cover non-exclusive inclusions, thereby causing a process, method, article, or apparatus that includes a series of elements to include not only those expressly listed elements, but also other elements not explicitly listed, or further include elements inherent to such process, method, article, or apparatus. In the absence of more limitations, an element defined by the phrase “comprising a . . . ” does not preclude the existence of additional identical elements in the process, method, article, or apparatus that includes the element. Furthermore, it should be noted that the scope of the methods and apparatus in the examples of the present application is not limited to performing functions in the order shown or discussed, and may further include performing functions substantially concurrently or in reverse order based on the related functions. For example, the described methods may be performed in an order different from that described, and may further include adding, omitting, or combining various steps. Additionally, features described with reference to certain embodiments may be combined in other embodiments.

The present invention provides a method for synthesizing a cyclic ketone compound from a diol and a ketone. The method includes: a ketone and a diol are employed as reaction substrates, cyclization is performed by coupling the α-carbon of the carbonyl group in the ketone with the α-carbon of the hydroxyl group in the diol under catalysis of a transition metal catalyst, and a novel cyclic ketone compound is formed.

Specifically, the method for synthesizing the cyclic ketone compound from the diol and the ketone includes the steps of:
S1. at least one diol is added into a cleaned reactor;
S2. at least one ketone is added into the reactor in S1;
S3. the transition metal catalyst is added into the reactor in S2; and
S4. the reactor is sealed, a temperature of the reactor is raised to 80-400° C., the reaction is performed under stirring for 0.5-8 h, and then the reaction product is filtered to obtain the cyclic ketone compound.

As some examples of the present invention, in S1, the diol is selected from at least one of ethylene glycol, 1,2-propanediol, 1,3-propanediol, 1,2-butanediol, 1,4-butanediol, 1,2-pentanediol, 1,5-pentanediol, 1,2-hexanediol, 1,6-hexanediol, 1,2-heptanediol, 1,7-heptanediol, 1,2-octanediol, 1,8-octanediol, 1,2-nonanediol, 1,9-nonanediol, 1,2-decanediol, and 1,10-decanediol.

As some examples of the present invention, in S2, the ketone in the reaction substrates is selected from at least one of acetone, butanone, 2-pentanone, 2-hexanone, 2-heptanone, 2-octanone, 2-nonanone, 2-decanone, and 2-undecanone.

Preferably, the transition metal catalyst is a supported transition metal catalyst.

As some examples of the present invention, the carrier in the supported transition metal catalyst may be selected from: (a) polymeric porous materials, such as polyolefin powders; (b) natural minerals, such as diatomaceous earth, kaolin, natural zeolite, expanded perlite and the like; and (c) ceramics, graphene, activated carbon, silica gel, or porous molecular sieves.

Preferably, the supported transition metal catalyst includes at least one transition metal element selected from Group VIIB, Group VIII, Group IB, and Group IIB of the periodic table, wherein the transition metal is a non-precious metal element.

More preferably, the transition metal in the supported transition metal catalyst is selected from Group 4 transition elements in the periodic table, including but not limited to manganese (Mn), iron (Fe), cobalt (Co), and nickel (Ni).

Further, the amount of the transition metal catalyst is 1% to 5% by weight relative to the total weight of the diol and ketone substrates.

Preferably, the reactor for synthesis is a high-pressure reactor.

Preferably, in S4, a temperature of the reactor is 150-260° C.

Further, in the added reaction substrates, a molar ratio of the diol to the ketone is in a range of (1:50)≤(n1:n2)≤(20:1), wherein the number of moles of the diol is n1, and the number of moles of the ketone is n2.

Preferably, in the added reaction substrates, the molar ratio of the diol to the ketone (n1:n2) ranges from 1:10 to 10:1.

More preferably, in the added reaction substrates, the molar ratio of diol to ketone (n1:n2) is 1:1.

Preferably, during the reaction process of S4, sampling may be performed through a sampling valve to monitor the reaction progress or degree of completion.

As some examples of the present invention, the prepared cyclic ketone compound includes cyclopentanone and cyclopentanone with R-group substituents on the side chain, cyclohexanone and cyclohexanone with R-group substituents on the side chain, cycloheptanone and cycloheptanone with R-group substituents on the side chain, cyclononanone and cyclononanone with R-group substituents on the side chain, as well as higher-membered cyclic ketones and derivatives with R-group substituents on the side chain, wherein R is a C1-C9 alkyl group

The present invention provides a condensation-coupling reaction between a diol and a ketone under the action of a catalyst, wherein the ketone and the diol are combined, and the α-C of the carbonyl group in the ketone couples with the α-C of the hydroxyl group in the diol to undergo cyclization, thereby obtaining a target cyclic ketone compound with high selectivity.

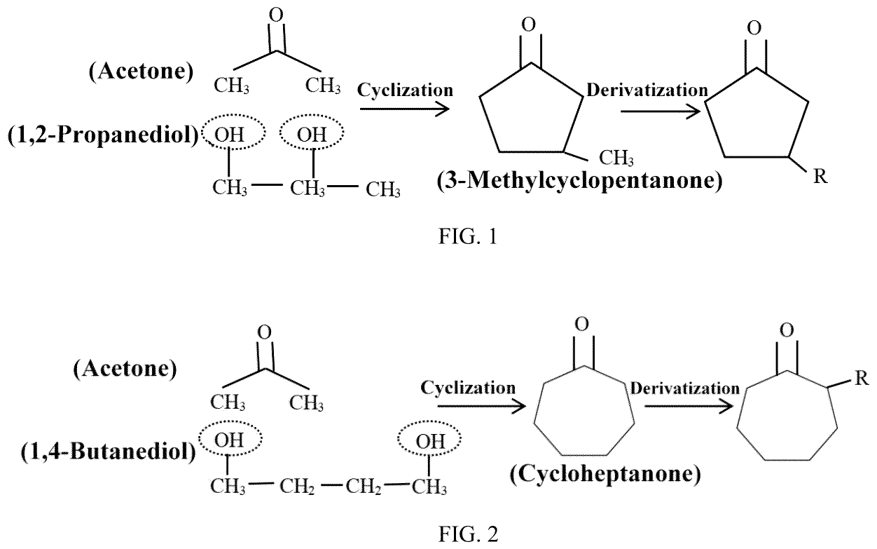

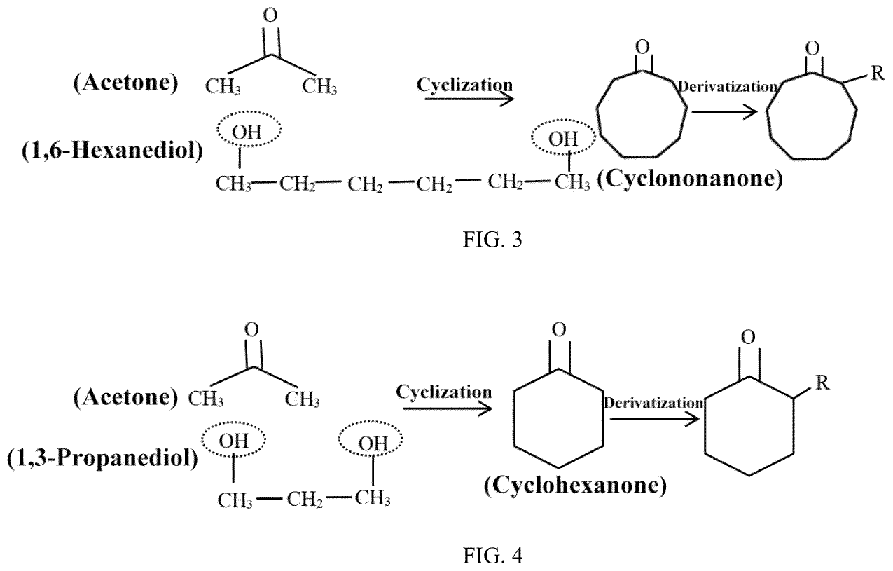

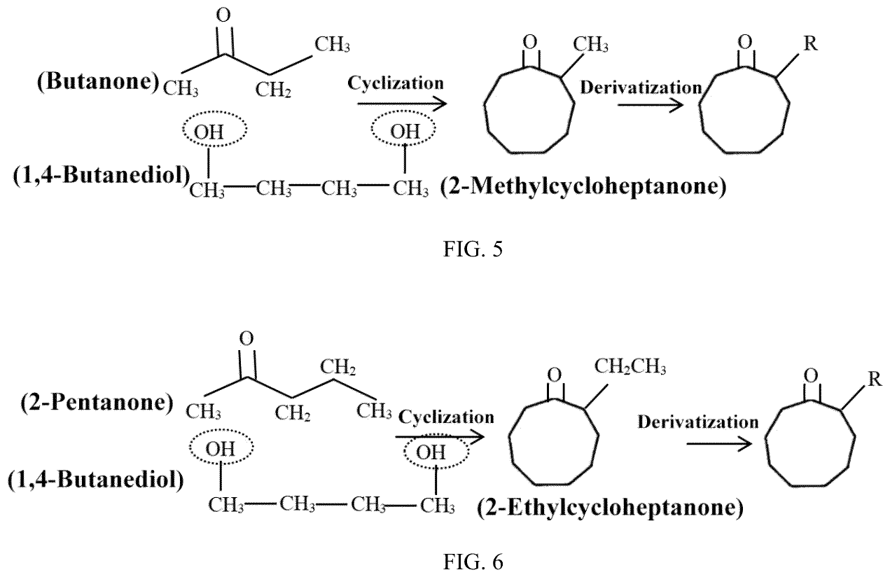

FIGS. 1-6 illustrate processes wherein different ketones and diols are used as reaction substrates to undergo cyclization via coupling between the α-C of the carbonyl group in the ketone and the α-C of the hydroxyl group in the diol under the action of a transition metal catalyst, thereby forming novel cyclic ketone compounds. Additionally, FIGS. 1-6 further indicate derivatives structurally similar to the generated cyclic ketone compounds, such as a methods synthesizing cyclopentanone derivatives featuring R-group substituted side chains structurally analogous to 3-methylcyclopentanone.

The method for synthesizing the cyclic ketone compound according to the present invention operates without external solvents or high-pressure hydrogen, featuring easily controllable reaction conditions and simplified preparation procedures.

Furthermore, the method demonstrates a high conversion rate for the condensation coupling and is applicable to a wide range of diols and ketones. Under preferred conditions, the conversion rates of the diol and the ketone may each reach over 90%. The target cyclic ketone compound may achieve a selectivity of over 90% and maintain stable operation over extended periods, demonstrating excellent industrial applicability.

The present invention is further illustrated by the following specific examples, which are not to be construed as limiting the scope of the invention. Specifically:

### Example 1

15.218 g of 1,2-propanediol and 11.616 g of acetone were charged into a cleaned high-pressure reactor, followed by addition of 0.805 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 210° C. with the stirring speed set to 500 r/min. After 4.5 h of reaction, the sampling valve was opened to collect a sample, which was identified as the reaction product. The reaction product was filtered, and the filtrate was collected for analysis. Based on 1,2-propanediol as the reference, a conversion rate of 92.363% was determined, with 3-methylcyclopentanone exhibiting a selectivity of 97.374%.

### Example 2

15.218 g of 1,2-propanediol and 14.421 g of butanone were charged into a cleaned high-pressure reactor, followed by addition of 0.889 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 210° C. with the stirring speed set to 500 r/min. After 5.2 h of reaction, the sampling valve was opened to collect a sample, which was identified as the reaction product. The reaction product was filtered, and the filtrate was collected for analysis. Based on 1,2-propanediol as the reference, a conversion rate of 94.325% was determined, with 2,3-dimethylcyclopentanone exhibiting a selectivity of 96.373%.

### Example 3

15.218 g of 1,2-propanediol and 17.226 g of 2-pentanone were charged into a cleaned high-pressure reactor, followed by addition of 0.973 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 210° C. with the stirring speed set to 500 r/min. After 4.3 h of reaction, the sampling valve was opened to collect a sample, which was identified as the reaction product. The reaction product was filtered, and the filtrate was collected for analysis. Based on 1,2-propanediol as the reference, a conversion rate of 93.579% was determined, with 3-methyl-2-ethylcyclopentanone exhibiting a selectivity of 96.519%.

### Example 4

18.024 g of 1,4-butanediol and 11.616 g of acetone were charged into a cleaned high-pressure reactor, followed by addition of 0.889 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 260° C. with the stirring speed set to 500 r/min. After 6 h of reaction, the sampling valve was opened to collect a sample, which was identified as the reaction product. The reaction product was filtered, and the filtrate was collected for analysis. Based on 1,4-butanediol as the reference, a conversion rate of 92.732% was determined, with cycloheptanone exhibiting a selectivity of 95.216%.

### Example 5

18.024 g of 1,4-butanediol and 14.421 g of butanone were charged into a cleaned high-pressure reactor, followed by addition of 0.973 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 260° C. with the stirring speed set to 500 r/min. After 6 h of reaction, the sampling valve was opened to collect a sample, which was identified as the reaction product. The reaction product was filtered, and the filtrate was collected for analysis. Based on 1,4-butanediol as the reference, a conversion rate of 97.368% was determined, with 2-methylcycloheptanone exhibiting a selectivity of 94.461%.

### Example 6

18.024 g of 1,4-butanediol and 17.226 g of 2-pentanone were charged into a cleaned high-pressure reactor, followed by addition of 1.057 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 260° C. with the stirring speed set to 500 r/min. After 4 h of reaction, the sampling valve was opened to collect a sample, which was identified as the reaction product. The reaction product was filtered, and the filtrate was collected for analysis. Based on 1,4-butanediol as the reference, a conversion rate of 95.647% was determined, with 2-ethylcycloheptanone exhibiting a selectivity of 93.612%.

### Example 7

23.634 g of 1,6-hexanediol and 11.616 g of acetone were charged into a cleaned high-pressure reactor, followed by addition of 1.058 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 260° C. with the stirring speed set to 500 r/min. After 3.5 h of reaction, the sampling valve was opened to collect a sample, which was identified as the reaction product. The reaction product was filtered, and the filtrate was collected for analysis. Based on 1,6-hexanediol as the reference, a conversion rate of 93.265% was determined, with cyclononanone exhibiting a selectivity of 91.364%.

### Example 8

23.634 g of 1,6-hexanediol and 14.421 g of butanone were charged into a cleaned high-pressure reactor, followed by addition of 1.142 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 260° C. with the stirring speed set to 500 r/min. After 5.5 h of reaction, the sampling valve was opened to collect a sample, which was identified as the reaction product. The reaction product was filtered, and the filtrate was collected for analysis. Based on 1,6-hexanediol as the reference, a conversion rate of 96.732% was determined, with 2-methylcyclononanone exhibiting a selectivity of 93.641%.

### Example 9

23.634 g of 1,6-hexanediol and 17.226 g of 2-pentanone were charged into a cleaned high-pressure reactor, followed by addition of 1.226 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 260° C. with the stirring speed set to 500 r/min. After 8 h of reaction, the sampling valve was opened to collect a sample, which was identified as the reaction product. The reaction product was filtered, and the filtrate was collected for analysis. Based on 1,6-hexanediol as the reference, a conversion rate of 96.376% was determined, with 2-ethylcyclononanone exhibiting a selectivity of 97.623%.

### Example 10

15.219 g of 1,3-propanediol and 11.616 g of acetone were charged into a cleaned high-pressure reactor, followed by addition of 0.805 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 230° C. with the stirring speed set to 500 r/min. After 7 h of reaction, the sampling valve was opened to collect a sample, which was identified as the reaction product. The reaction product was filtered, and the filtrate was collected for analysis. Based on 1,3-propanediol as the reference, a conversion rate of 94.336% was determined, with cyclohexanone exhibiting a selectivity of 96.419%.

### Example 11

15.219 g of 1,3-propanediol and 14.421 g of butanone were charged into a cleaned high-pressure reactor, followed by addition of 0.889 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 230° C. with the stirring speed set to 500 r/min. After 3 h of reaction, the sampling valve was opened to collect a sample, which was identified as the reaction product. The reaction product was filtered, and the filtrate was collected for analysis. Based on 1,3-propanediol as the reference, a conversion rate of 91.325% was determined, with 2-methylcyclohexanone exhibiting a selectivity of 91.251%.

### Example 12

15.219 g of 1,3-propanediol and 17.226 g of 2-pentanone were charged into a cleaned high-pressure reactor, followed by addition of 0.973 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 230° C. with the stirring speed set to 500 r/min. After 3 h of reaction, the sampling valve was opened to collect a sample, which was identified as the reaction product. The reaction product was filtered, and the filtrate was collected for analysis. Based on 1,3-propanediol as the reference, a conversion rate of 94.343% was determined, with 2-ethylcyclohexanone exhibiting a selectivity of 91.244%.

### Example 13

0.3 g of 1,2-propanediol and 11.616 g of acetone were charged into a cleaned high-pressure reactor, followed by addition of 0.142 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 150° C. with the stirring speed set to 500 r/min. After 1 h of reaction, the sampling valve was opened to collect a sample, which was identified as the reaction product. The reaction product was filtered, and the filtrate was collected for analysis. Based on 1,2-propanediol as the reference, a conversion rate of 97.43% was determined, with 3-methylcyclopentanone exhibiting a selectivity of 98.561%.

### Example 14

15.218 g of 1,2-propanediol and 0.581 g of acetone were charged into a cleaned high-pressure reactor, followed by addition of 0.158 g of an activated carbon-supported nickel-based catalyst into the same reactor. The high-pressure reactor was sealed, and the high-pressure reactor's sampling valve and gas charging valve were tightened. The high-pressure reactor was started, and the temperature was adjusted to 80° C. with the stirring speed set to 500 r/min. After 0.5 h of reaction, the sampling valve was opened to collect a sample, which was identified as the reaction product. The reaction product was filtered, and the filtrate was collected for analysis. Based on acetone as the reference, a conversion rate of 97.359% was determined, with 3-methylcyclopentanone exhibiting a selectivity of 96.712%.

### Example 15

1.522 g of 1,2-propanediol and 11.616 g of acetone were charged into a cleaned high-pressure reactor, followed by addition of 0.576 g of an activated carbon-supported cobalt-based catalyst into the same reactor. The high-pressure reactor was sealed, and the high-pressure reactor's sampling valve and gas charging valve were tightened. The high-pressure reactor was started, and the temperature was adjusted to 320° C. with the stirring speed set to 500 r/min. After 0.8 h of reaction, the sampling valve was opened to collect a sample, which was identified as the reaction product. The reaction product was filtered, and the filtrate was collected for analysis. Based on 1,2-propanediol as the reference, a conversion rate of 97.895% was determined, with 3-methylcyclopentanone exhibiting a selectivity of 98.423%.

### Example 16

15.218 g of 1,2-propanediol and 1.162 g of acetone were charged into a cleaned high-pressure reactor, followed by addition of 0.296 g of an activated carbon-supported iron-based catalyst into the same reactor. The high-pressure reactor was sealed, and the high-pressure reactor's sampling valve and gas charging valve were tightened. The high-pressure reactor was started, and the temperature was adjusted to 400° C. with the stirring speed set to 500 r/min. After 1 h of reaction, the sampling valve was opened to collect a sample, which was identified as the reaction product. The reaction product was filtered, and the filtrate was collected for analysis. Based on 1,2-propanediol as the reference, a conversion rate of 98.768% was determined, with 3-methylcyclopentanone exhibiting a selectivity of 98.169%.

In summary, the present invention provides a method for synthesizing a cyclic ketone compound from a diol and a ketone. The method includes adding a supported transition metal catalyst, diol, and ketone into a high-pressure reactor, controlling reaction temperature and agitation speed, via a highly efficient catalytic condensation-coupling reaction between the ketone and a small-molecule alcohol, to obtain a target cyclic ketone with high selectivity. In the method for synthesizing the cyclic ketone compound from the diol and the ketone according to the present invention, after adding the reactants, the reaction proceeds without requiring an external solvent or high-pressure hydrogen, making the process easy to implement with a straightforward preparation method. Furthermore, the method for synthesizing the cyclic ketone compound from the diol and the ketone according to the present invention is applicable to a broad range of diol and ketone substrates, with conversion rates exceeding 90% for both the diol and the ketone. The target cyclic ketone compound is obtained with a selectivity of at least 90%, and the process demonstrates long-term operational stability, exhibiting excellent potential for industrial application.

The examples of the present application have been described above in conjunction with the accompanying drawings. Without conflict, the examples and features in the examples of the present application may be combined with each other. The present application is not limited to the specific embodiments described above, which are merely illustrative and not restrictive. Those skilled in the art may derive various modifications based on the disclosure of the present application without departing from the spirit and scope of the present application as defined by the claims, all of which shall fall within the protection scope of the present application.

What is claimed is:

1. A method for synthesizing a cyclic ketone compound from a diol and a ketone, comprising: employing the ketone and the diol as reaction substrates, and cyclizing by coupling an α-carbon of a carbonyl group in the ketone with an α-carbon of a hydroxyl group in the diol under a catalysis of a transition metal catalyst, to form the cyclic ketone compound.

2. The method for synthesizing the cyclic ketone compound from the diol and the ketone according to claim 1, wherein the method comprises steps of:
S1, adding at least one diol into a cleaned reactor;
S2, adding at least one ketone into a reactor in the step S1;
S3, adding the transition metal catalyst into a reactor in the step S2; and
S4, sealing a reactor in the step S3 to obtain a sealed reactor, raising a temperature of the sealed reactor to 80-400° C., reacting under stirring for 0.5-8 h, and then filtering a reaction product to obtain the cyclic ketone compound.

3. The method for synthesizing the cyclic ketone compound from the diol and the ketone according to claim 1, wherein the diol is selected from at least one of ethylene glycol, 1,2-propanediol, 1,3-propanediol, 1,2-butanediol, 1,4-butanediol, 1,2-pentanediol, 1,5-pentanediol, 1,2-hexanediol, 1,6-hexanediol, 1,2-heptanediol, 1,7-heptanediol, 1,2-octanediol, 1,8-octanediol, 1,2-nonanediol, 1,9-nonanediol, 1,2-decanediol, and 1,10-decanediol.

4. The method for synthesizing the cyclic ketone compound from the diol and the ketone according to claim 1, wherein the ketone in the reaction substrates is selected from at least one of acetone, butanone, 2-pentanone, 2-hexanone, 2-heptanone, 2-octanone, 2-nonanone, 2-decanone, and 2-undecanone.

5. The method for synthesizing the cyclic ketone compound from the diol and the ketone according to claim 1, wherein the transition metal catalyst is a supported transition metal catalyst.

6. The method for synthesizing the cyclic ketone compound from the diol and the ketone according to claim 5, wherein a transition metal in the supported transition metal catalyst comprises at least one transition metal element selected from Group VIIB, Group VIII, Group IB, and Group IIB of periodic table, wherein the transition metal is a non-precious metal element.

7. The method for synthesizing the cyclic ketone compound from the diol and the ketone according to claim 2, wherein in the step S4, the temperature of the sealed reactor is 150-260° C.

8. The method for synthesizing the cyclic ketone compound from the diol and the ketone according to claim 1, wherein in the reaction substrates, a molar ratio of the diol to the ketone is in a range of (1:50)≤(n1:n2)≤(20:1), wherein a number of moles of the diol is n1, and a number of moles of the ketone is n2.

9. The method for synthesizing the cyclic ketone compound from the diol and the ketone according to claim 2, wherein the cleaned reactor for a synthesis is a high-pressure reactor.

10. The method for synthesizing the cyclic ketone compound from the diol and the ketone according to claim 1, wherein the cyclic ketone compound comprises cyclopentanone and cyclopentanone with R-group substituents on a side chain of the cyclopentanone, cyclohexanone and cyclohexanone with R-group substituents on a side chain of the cyclohexanone, cycloheptanone and cycloheptanone with R-group substituents on a side chain of the cycloheptanone, cyclononanone and cyclononanone with R-group substituents on a side chain of the cyclononanone, and higher-membered cyclic ketones and derivatives with R-group substituents on a side chain of the higher-membered cyclic ketones, wherein R is a C1-C9 alkyl group.

11. The method for synthesizing the cyclic ketone compound from the diol and the ketone according to claim 2, wherein the diol is selected from at least one of ethylene glycol, 1,2-propanediol, 1,3-propanediol, 1,2-butanediol, 1,4-butanediol, 1,2-pentanediol, 1,5-pentanediol, 1,2-hexanediol, 1,6-hexanediol, 1,2-heptanediol, 1,7-heptanediol, 1,2-octanediol, 1,8-octanediol, 1,2-nonanediol, 1,9-nonanediol, 1,2-decanediol, and 1,10-decanediol.

12. The method for synthesizing the cyclic ketone compound from the diol and the ketone according to claim 2, wherein the ketone in the reaction substrates is selected from at least one of acetone, butanone, 2-pentanone, 2-hexanone, 2-heptanone, 2-octanone, 2-nonanone, 2-decanone, and 2-undecanone.

13. The method for synthesizing the cyclic ketone compound from the diol and the ketone according to claim 2, wherein the transition metal catalyst is a supported transition metal catalyst.

14. The method for synthesizing the cyclic ketone compound from the diol and the ketone according to claim 2, wherein in the reaction substrates, a molar ratio of the diol to the ketone is in a range of (1:50)≤(n1:n2)≤(20:1), wherein a number of moles of the diol is n1, and a number of moles of the ketone is n2.

15. The method for synthesizing the cyclic ketone compound from the diol and the ketone according to claim 13, wherein a transition metal in the supported transition metal catalyst comprises at least one transition metal element selected from Group VIIB, Group VIII, Group IB, and Group IIB of periodic table, wherein the transition metal is a non-precious metal element.
